(19) United States
(12) Patent Application Publication
(10) Pub. No.: US 2026/0207682 A1
(43) Pub. Date: Jul. 23, 2026

(54) PROBIOTIC FORMULATIONS AND METHODS OF USE THEREOF

(71) Applicant: NewGen 27, LLC d/b/a Doctor's Biome, Westbury, NY (US)

(72) Inventors: Howard F. Robins, Westbury, NY (US); A. Reza Kamarei, Princeton, NJ (US); Richard Finkelstein, Long Beach, NY (US); Linette Robins, Westbury, NY (US); Eric Finkelstein, Ridgefield, CT (US)

(21) Appl. No.: 19/129,769

(22) PCT Filed: Nov. 15, 2023
(86) PCT No.: PCT/US2023/079812

§ 371 Date: May 14, 2025

Related U.S. Application Data
(60) Provisional application No. 63/425,387, filed on Nov. 15, 2022.

(51) Int. Cl. A61K 35/745; A23L 2/02; A23L 2/52; A23L 33/00; A61K 9/00; A61K 35/00; A61K 35/747; A61P 31/04

(52) U.S. Cl. CPC A61K 35/745; A23L 2/02; A23L 2/52; A23L 33/30; A61K 9/0095; A61K 35/747; A61P 31/04; A61K 2035/115

(57) ABSTRACT

The present invention relates to a probiotic formulation comprising a blend of strains of living Bifidobacterium and Lactobacillus species in a sterilized liquid fruit and vegetable juice medium. The blend of the bacterial strains release bioactive compounds in the sterilized juice medium. Upon ingestion of the sterilized juice medium, in addition to competitive exclusion of C. difficile and/or C. perfringens, the bioactive compounds lower the pH of the individual's gastro-intestinal tract to an acidic range making it unfavorable for spore formation and/or growth of C. difficile and/or C. perfringens.

### TECHNICAL FIELD OF THE INVENTION

The present invention generally refers to probiotic formulations and methods to use the probiotic formulation to inhibit the growth of harmful and pathogenic organisms in the human gastro-intestinal tract.

### BACKGROUND OF THE INVENTION

Microbiome is the genome of micro-organisms in a particular environment, while microbiota refers to a community of micro-organisms themselves. Approximately, 100 trillion micro-organisms exist in the gastrointestinal tract of a human being. (Valdes A M, Walter J, Segal E, Spector T D. Role of the gut microbiota in nutrition and health. BMJ. 2018 Jun. 13; 361: k2179. doi: 10.1136/bmj.k2179. PMID: 29899036; PMCID: PMC6000740). Thus, the human gastro-intestinal tract is home to a large population of microbiota. The number of micro-organisms lining the gastro-intestinal tract has been estimated to be 10 times more than the number of human cells. Further, microbial genes outnumber human genes by a factor of 100. Gastro-intestinal microbiota produce energy by metabolization of complex polysaccharides and dietary fibers which are otherwise not digested by humans. Metabolization of such non-digestible substrates result in the generation of various metabolites including short chain fatty acids such as acetate, propionate, and butyrate which help in activation of intestinal gluconeogenesis and can induce apoptosis of colon cancer cells.

Gastro-intestinal microbiota are crucial in many aspects of human health including metabolic, mental, as well as physiological health. Disruption and alteration of the gastro-intestinal microbiota has been recognized in several disorders such as antibiotic associated diarrhea. Antibiotic associated diarrhea is caused due to Clostridium difficile (C. difficile).

C. difficile is a Gram-positive, spore-forming bacterium responsible for infection of the colon. C. difficile infection (CDI) is one of the most prevalent nosocomial (hospital-acquired) infections. C. difficile has been shown to infect individuals aged 65 years or older. Further, more than 100,000 C. difficile infections develop among individuals residing in nursing homes in the United States. CDI generally occurs after use of antibiotics which may disturb microbiota in the colon. A disturbed microbiota in the colon leads to loss of colonization resistance, causing vulnerability to CDI, which can be fatal.

Clostridium perfringens (C. perfringens) is a Gram-positive, bacillus (rod-shaped), anaerobic, spore-forming pathogenic bacterium. Clostridium perfringens is ever-present in nature and can be found as a normal component of the intestinal tract of humans. Clostridium perfringens bacteria are one of the most common causes of food poisoning. Center for Disease Control and Prevention estimates that the bacterium causes nearly 1 million foodborne illnesses in the United States every year. Most people with C. perfringens food poisoning have diarrhea and stomach cramps but no vomiting. Symptoms usually begin 6 to 24 hours after swallowing the bacteria. Symptoms can start suddenly and usually last for less than 24 hours. C. perfringens food poisoning is diagnosed when a laboratory test detects the bacteria or toxin in a patient's stool sample or the bacteria are found in food linked to the illness. Most people recover from C. perfringens food poisoning without antibiotics.

There is a need for approaches to restore as well as preserve gastro-intestinal microbiota. There has been substantial research regarding use of various probiotic strains against C. difficile and C. perfringens. However, none of the research generated consistent results. Therefore, probiotic formulations that inhibits growth and pathogenicity of C. difficile and C. perfringens is necessary.

### SUMMARY OF THE INVENTION

The present invention is directed to novel probiotic formulations that improve gut health in individuals with Clostridium difficile infection (CDI) and/or Multiple Sclerosis (MS) caused by epsilon-toxin producing C. perfringens.

The first aspect of the invention provides a probiotic formulation comprising a liquid medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures.

Probiotic formulations of the invention comprise at least one species from the genus Bifidobacterium and at least one species from the genus Lactobacillus. Probiotic formulations of the invention may comprise each species in the liquid medium from B. bifidum, B. breve, B. lactis, B. longum, B. infantis, and L. acidophilus, L. brevis, L. bulgaricus, L. casei, L. gasseri, L. paracasei, L. plantarum, L. reuteri, L. rhamnosus, and L. salivarius. For example, each species may be present in substantially the same percentage. The probiotic formulations may further comprise a no-added sugar content within predetermined concentration range of about 5% to about 15% (for example, 5%, 6%, 7%, 8%, 9%, 10%, 11%, 12%, 13%, 14%, or 15%).

Probiotic formulations of the invention may further comprise a sterilized juice medium. The sterilized juice medium may be a sterilized liquid fruit and vegetable juice medium. The sterilized liquid fruit and vegetable juice medium may be at least one of or a mixture of mint, cucumber, apple, lettuce, kale, celery, and lemon. The sterilized liquid juice medium used in the probiotic formulation of the present invention is organic and devoid of pesticides, chemicals or additives of any sort.

The probiotic formulation of the present invention may be in the form of a drinkable juice beverage which has a desirable shelf-life without the need of refrigeration, yet maintaining the viability of the bacterial strains. The probiotic formulation may also include a prebiotic.

The strains forming a part of the probiotic formulation of the first aspect of the invention may be actively living and pre-hydrated. The probiotic formulation of the present invention may comprise from 1 to about 200 billion colony forming units of the said actively living and pre-hydrated strains in a 2 fluid ounce bottle of juice beverage at the time of manufacture of the said beverage. Advantageously, the formulation may be a dietary supplement or a medical food.

The living probiotic bacterial strains of the formulation release bioactive compounds into the sterilized liquid fruit and vegetable juice medium, which help reduce the pH of a user's gut to an acidic range. Acidic pH of the user's gut is unfavorable for spore germination and/or growth of Clostridium difficile (C. difficile) and/or C. perfringens. The probiotic formulation thereby helps improve or mitigate C. difficile infection in a user's gut and/or MS.

The second aspect of the present invention provides a method for improving gut health of an individual with C. difficile infection, where the method comprises administering to an individual having C. difficile infection, the probiotic formulation. The probiotic formulation comprises a liquid medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures.

Probiotic formulations of the invention comprise at least one species from the genus Bifidobacterium and at least one species from the genus Lactobacillus. Probiotic formulations of the invention may comprise each species in the liquid medium from B. bifidum, B. breve, B. lactis, B. longum, B. infantis, and L. acidophilus, L. brevis, L. bulgaricus, L. casei, L. gasseri, L. paracasei, L. plantarum, L. reuteri, L. rhamnosus, and L. salivarius. For example, each species may be present in substantially the same percentage. The probiotic formulations may further comprise a no-added sugar content within predetermined concentration range of about 5% to about 15% (for example, 5%, 6%, 7%, 8%, 9%, 10%, 11%, 12%, 13%, 14%, or 15%).

In methods of the invention, the liquid medium may comprise a sterilized juice medium. The sterilized juice medium may comprise a sterilized liquid fruit and vegetable juice medium. The sterilized liquid fruit and vegetable juice medium may be at least one of or a mixture of mint, cucumber, apple, lettuce, kale, celery, and lemon. The sterilized liquid juice medium used in the probiotic formulation of the present invention is organic and devoid of pesticides, chemicals or additives of any sort.

The probiotic formulation of the present invention is in the form of a drinkable juice beverage which has a desirable shelf-life without the need of refrigeration, yet maintaining the viability of the bacterial strains. The probiotic formulation may also include a prebiotic.

In the methods of the invention, the strains forming a part of the probiotic formulation may be actively living and pre-hydrated. The probiotic formulation of the present invention may comprise from 1 to about 200 billion colony forming units of the said actively living and pre-hydrated strains in a 2 fluid ounce bottle of juice beverage at the time of manufacture of the said beverage. Advantageously, in methods of the invention, the formulation may be provided as a dietary supplement or a medical food.

The living probiotic bacterial strains of the method release bioactive compounds into the sterilized liquid fruit and vegetable juice medium, which help reduce the pH of a user's gut to an acidic range. Acidic pH of the user's gut is unfavorable for spore germination and/or growth of Clostridium difficile (C. difficile). The probiotic formulation thereby helps improve or mitigate C. difficile infection in a user's gut.

The third aspect of the present invention provides a method for inhibition or amelioration of multiple sclerosis (MS) in an individual, where the method comprises administering to an individual the probiotic formulation. The probiotic formulation comprises a liquid medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures.

Probiotic formulations of the invention comprise at least one species from the genus Bifidobacterium and at least one species from the genus Lactobacillus. Probiotic formulations of the invention may comprise each species in the liquid medium from B. bifidum, B. breve, B. lactis, B. longum, B. infantis, and L. acidophilus, L. brevis, L. bulgaricus, L. casei, L. gasseri, L. paracasei, L. plantarum, L. reuteri, L. rhamnosus, and L. salivarius. For example, each species may be present in substantially the same percentage. The probiotic formulations may further comprise a no-added sugar content within predetermined concentration range of about 5% to about 15% (for example, 5%, 6%, 7%, 8%, 9%, 10%, 11%, 12%, 13%, 14%, or 15%).

In methods of the invention, the liquid medium may comprise a sterilized juice medium. The sterilized juice medium may comprise a sterilized liquid fruit and vegetable juice medium. The sterilized liquid fruit and vegetable juice medium may be at least one of or a mixture of mint, cucumber, apple, lettuce, kale, celery, and lemon. The sterilized liquid juice medium used in the probiotic formulation of the present invention is organic and devoid of pesticides, chemicals or additives of any sort.

The probiotic formulation of the present invention is in the form of a drinkable juice beverage which has a desirable shelf-life without the need of refrigeration, yet maintaining the viability of the bacterial strains. The probiotic formulation may also include a prebiotic.

In the methods of the invention, the strains forming a part of the probiotic formulation may be actively living and pre-hydrated. The probiotic formulation of the present invention may comprise from 1 to about 200 billion colony forming units of the said actively living and pre-hydrated strains in a 2 fluid ounce bottle of juice beverage at the time of manufacture of the said beverage. Advantageously, in methods of the invention, the formulation may be provided as a dietary supplement or a medical food.

The living probiotic bacterial strains of the method release bioactive compounds into the sterilized liquid fruit and vegetable juice medium, which help reduce the pH of a user's gut to an acidic range. Acidic pH of the user's gut is unfavorable for spore germination and/or growth of C. perfringens. The probiotic formulation thereby helps prevent growth of epsilon-toxin producing C. perfringens, thereby inhibiting or ameliorating MS.

### DETAILED DESCRIPTION OF THE INVENTION

By the way of overview, the present invention discloses a probiotic formulation and a method for improving gut health of an individual with Clostridium difficile (C. difficile) or Clostridium perfringens (C. perfringens) infection. In particular, the invention provides a juice beverage, comprising living probiotic bacterial strains in a predetermined range. The juice beverage comprising living probiotic bacterial strains is provided to an individual with C. difficile infection or C. perfringens infection, thereby improving the individual's gut health

### Clostridium Difficile

Clostridium difficile also called as C. difficile is a spore forming, motile, anaerobic, Gram-positive bacteria. C. difficile bacterium can cause uncontrolled diarrhea which can be life-threatening. Clostridium difficile infection (CDI) is among the most prevalent nosocomial infections caused due to adverse effects of antibiotics. Symptoms of CDI include diarrhea, watery stools lasting for several days, tenderness of stomach, fever, nausea. CDI is common in individuals with weakened immune systems or with previous infection with C. difficile. Disturbance of the gastro-intestinal microbiota play an important role in the pathogenesis of C. difficile. A diverse community of micro-organisms colonize the gastro-intestinal tract. This diverse community of gastro-intestinal micro-organisms, prevent the colonization of the gastro-intestinal tract by undesirable and potentially harmful micro-organisms. In order to protect itself from injury as well as maintain homeostasis, the gastro-intestinal tract recruits intestinal barriers such as physical, biochemical, and immunological. Physical barriers include mucus layers, biochemical barriers include enzymes, while immunological barriers include immunoglobulin A (IgA).

Several factors can substantially cause an imbalance of the gastrointestinal micro-organisms including hospitalization, diet, genetics, and use of medications, especially antibiotics. Disturbance of gastro-intestinal micro-organisms may lead to various conditions such as irritable bowel syndrome, rheumatoid arthritis, diabetes, inflammatory bowel syndrome, auto-immune diseases, depression, anxiety, autism, allergies, diarrhea including diarrhea induced by antibiotics as well as bacterial and/or viral diarrhea, and infection by C. difficile.

Maintaining a balance of the gastro-intestinal micro-organisms is a fundamental step in combating undesirable health conditions including C. difficile infection.

Closterium perfringens

Clostridium perfringens (C. perfringens) is a Gram-positive, bacillus (rod-shaped), anaerobic, spore-forming pathogenic bacterium. Clostridium perfringens is ever-present in nature and can be found as a normal component of the intestinal tract of humans. Clostridium perfringens bacteria are one of the most common causes of food poisoning. Center for Disease Control and Prevention estimates that the bacterium causes nearly 1 million foodborne illnesses in the United States every year. Most people with C. perfringens food poisoning have diarrhea and stomach cramps but no vomiting. Symptoms usually begin 6 to 24 hours after swallowing the bacteria. Symptoms can start suddenly and usually last for less than 24 hours. C. perfringens food poisoning is diagnosed when a laboratory test detects the bacteria or toxin in a patient's stool sample or the bacteria are found in food linked to the illness. Most people recover from C. perfringens food poisoning without antibiotics.

There are five toxin types (A, B, C, D and E) of C. perfringens based upon the production of one or more major protein toxins. These toxins are linked to diverse diseases/intoxications of humans and/or animals. See Stiles et al, “Clostridium perfringens Epsilon Toxin: A Malevolent Molecule for Animals and Man” Toxins 2013, 5, 2138-2160; doi: 10.3390/toxins5112138.

Major Toxins for C. perfringens Typing

| | C. perfringens Type | | | | | Cellular Target |
|---|---|---|---|---|---|---|
| Toxin | A | B | C | D | E | (mode of action) |
| Alpha | + | + | + | + | + | Membrane (phospholipid |
| | | | | | | destruction) |
| Beta | | + | + | | | Membrane (pore formation) |
| Epsilon | | + | | + | | Membrane (pore formation) |
| Iota | | | | | + | Actin (cytoskeleton destruction) |

### Multiple Sclerosis and C. Perfringens

Multiple sclerosis (MS) is a disease that impacts the brain, spinal cord and optic nerves, which make up the central nervous system. The exact cause of MS is unknown, however MS results in the immune system attacking the bodies central nervous system. The resulting damage to myelin, the protective layer insulating wire-like nerve fibers, disrupts signals to and from the brain. This interruption of communication signals causes unpredictable symptoms such as numbness, tingling, mood changes, memory problems, pain, fatigue, blindness and/or paralysis. Everyone's experience with MS is different and these losses may be temporary or long lasting.

Currently, multiple sclerosis is classified into four types or disease courses:
Clinically Isolated Syndrome (CIS)—the first episode of MS symptom
Relapsing-Remitting MS (RRMS)—the most common form of multiple sclerosis. Flare-ups (relapse or exacerbation) of new or worsening symptoms followed by periods of remission (when symptoms stabilize or go away).
Primary Progressive MS (PPMS)—Symptoms slowly and gradually worsen without any periods of relapse or remission.
Secondary Progressive MS (SPMS)—people originally diagnosed with RRMS eventually progress to SPMS. Symptoms progressively worsen. While person may still experience some relapses or flares, person no longer has periods of remission afterward.

Recent insights have shown that MS is a complex disease and is thought to require an environmental trigger. Although gut dysbiosis is common in MS, the specifically causative species are unknown. Sensitive and quantitative PCR detection has shown that individuals with MS were more likely to harbor and show a greater abundance of epsilon toxin (ETX)-producing strains of C. perfringens within their gut microbiomes compared to healthy controls. Accordingly, it is thought that ETX-producing C. perfringens strains may be biologically pathogens in MS that trigger inflammatory demyelination in the context of circulating myelin autoreactive lymphocytes. See Ma (2023) J Clin Invest. 133 (9): e163239 and U.S. Pat. No. 9,758,573, the contents of each of which are incorporated by reference herein in their entirety.

### Probiotics

Probiotics have proven to be vital to preserve as well as replenish helpful gastro-intestinal micro-organisms. Probiotics are live micro-organisms that, when administered in adequate quantities, offer an array of health benefits to the host. The health benefits include reducing antibiotic-associated diarrhea, improving lactose intolerance, improving allergies, and treating infectious diarrhea. Probiotics available are marketed in several forms. For example, probiotics may be marketed in a dry form or in the form of a liquid. Probiotics in a dry form are not physiologically active. In order to make the dry probiotic cells physiologically active again, the probiotics are suspended in an aqueous environment in the presence of water and nutrients. The probiotic dry cells may acquire nutrition from a prebiotic. Prebiotics support probiotic bacteria by inducing their growth and activity. Prebiotics are compounds found in food that induce the growth of these beneficial probiotic micro-organisms. Dietary prebiotics are non-digestible fiber that stimulate and facilitate the growth of beneficial gut microbiota. On the other hand, probiotics in a liquid form are physiologically active as they are suspended in an aqueous medium, and have access to the nutrition present in the medium.

Among the wide range of probiotic micro-organisms, Bifidobacterium and Lactobacillus are recognized for being beneficial to gut health. Bifidobacterium species are Gram-positive, non-motile inhabitants of the human gastro-intestinal tract. Lactobacillus species constitute a substantial portion of the human gastro-intestinal system, allowing the gastro-intestinal to persist severe environments. Lactobacillus species are Gram-positive bacteria which exist in a synergistic relationship with the host, as they shield the host from potential pathogenic invasions, and in turn, receive nutrients from the host.

Accordingly, first aspect of the present invention is directed to a probiotic formulation for oral administration. The probiotic formulation of the present invention is a liquid juice medium comprising one or more strains of living probiotic bacteria. Any living probiotic bacteria suitable for human administration may be used. For example, probiotic bacterial strains may be selected from the genus Lactobacillus or Bifidobacterium. In one embodiment, the living bacterial strains that may be used include, but are not limited to B. animalis, B. adolescentis, B. bifidum, B. breve, B. lactis, B. longum, B. infantis, L. acidophilus, L. brevis, L. bulgaricus, L. casei, L. crispatus, L. fermentum, L. helveticus, L. lactis, L. gasseri, L. paracasei, L. plantarum, L. reuteri, L. rhamnosus, L. salivarius, S. thermophilus, E. faecium. In another embodiment, the probiotic formulation may include a combination of such living bacterial strains. In one embodiment, the probiotic formulation may be a blend of living bacterial strains of B. bifidum, B. breve, B. lactis, B. longum, B. infantis, L. acidophilus, L. brevis, L. bulgaricus, L. casei, L. gasseri, L. paracasei, L. plantarum, L. reuteri, L. rhamnosus, L. salivarius, and known as the Doctor's Biome Colon Health (DBCH), known by the trade name Doctor's Biome Colon Health. Each living bacterial strain present in the liquid juice medium is present in substantially the same percentage.

The probiotic formulation of the present invention may be in the form of a beverage suitable for oral administration. The probiotic formulation may be a dietary supplement or a medical food. The beverage may comprise living bacterial strains dispersed in a liquid juice medium. The beverage may be in the form of a sterilized liquid fruit and vegetable juice medium. The sterilized liquid fruit and vegetable juice medium may include, but is not limited to, mint juice, cucumber juice, apple juice, lettuce juice, kale juice, celery juice, lemon juice, and mixtures thereof. The sterilized liquid fruit and vegetable juice medium may be organic and free of chemicals, pesticides, and/or additives. The probiotic formulation of the invention may include one or more prebiotics. Examples of prebiotics used in the probiotic formulation include but are not limited to inulin, maltodextrin, starch, and/or mixtures thereof. The formulation is a drinkable juice beverage with a desirable shelf-life while also maintaining viability of the bacterial strains without the need of refrigeration or cooling of the beverage.

The formulation may be a dietary supplement or a medical food. Dietary supplements include products taken orally that contain a “dietary ingredient” (vitamins, minerals, herbs or other botanicals, amino acids, and substances such as enzymes, organ tissues, glandulars, and metabolites) intended to supplement a diet. Dietary supplements also include extracts or concentrates, and may be found in many forms such as tablets, capsules, softgels, gelcaps, liquids, or powders. They can also be in other forms, such as a bar. Information may be included on the label of a dietary supplement must not represent the product as a conventional food or a sole item of a meal or diet.

Medical foods include a food which is formulated to be consumed or administered enterally under the supervision of a physician and which is intended for the specific dietary management of a disease or condition for which distinctive nutritional requirements, based on recognized scientific principles, are established by medical evaluation.

The probiotic formulation of the present invention may be formulated to include sugar at a predetermined concentration. In some embodiments, the probiotic formulation may comprise no-added sugar in the range of 5%-15%. For example, the probiotic formulation may comprise no-added sugar concentration of 5%, 6%, 7%, 8%, 9%, 10%, 11%, 12%, 13%, 14%, or 15%.

The probiotic formulation may further include excipients and/or additives, including but not limited to antioxidants and taste enhancers.

The probiotic formulation may contain living probiotic bacteria at a predetermined range of colony forming units. Colony forming units is a measure or a unit that provides an estimate of the number of viable microbial cells in a given sample.

Accordingly, the probiotic formulation may be formulated to provide between 1 billion and 200 billion colony forming units in a serving. The probiotic formulation may comprise between 1 billion and 200 billion colony forming units of pre-hydrated, actively living bacterial strains in the sterilized liquid fruit and vegetable juice medium. In one embodiment, there may be between 1 billion and 200 billion colony forming units of pre-hydrated, actively living bacterial strains in a 2-ounce bottle of the sterilized liquid fruit and vegetable juice medium.

The probiotic formulation may be useful for prophylaxis and/or mitigating or amelioration or treatment of disorders and infections including but not limited to bacterial and/or viral infections, irritable bowel syndrome, bloating, diarrhea including antibiotic associated diarrhea, Crohn's disease or ulcerative colitis, systemic diseases such as rheumatoid arthritis, and incomplete evacuation of bowels. Preferably, the infection treated by the probiotic formulation is C. difficile infection or C. perfringens infection. In some embodiments, the probiotic formulation may be used to improve gut health of an individual by inhibiting the growth of undesirable bacteria and pathogens. In some embodiments, the probiotic formulation may help ameliorate Clostridium difficile infection or C. perfringens infection.

In one embodiment, the probiotic formulation of the present invention is used to inhibit growth of undesirable bacteria and pathogens in an individual's gut. In another embodiment, the probiotic formulation helps ameliorate Clostridium difficile infection (CDI) or C. perfringens infection. The living probiotic bacterial strains present in the sterilized liquid fruit and vegetable juice medium release certain bioactive compounds into the medium. Upon oral administration of the sterilized liquid fruit and vegetable juice medium, the effect of bioactive compounds being released may reduce the pH of the user's gut to an acidic range which is unfavorable for spore germination and/or growth of C. difficile or C. perfringens.

Second aspect of the present invention is directed to a method of improving gut health of an individual with C. difficile or C. perfringens. The method is further directed to providing an individual with C. difficile or C. perfringens a probiotic formulation to reduce or inhibit growth of C. difficile or C. perfringens in the individual, thereby improving gut health of the individual. Preferably, the probiotic formulation is administered orally in the form of a liquid juice beverage. The probiotic formulation comprises a liquid juice medium comprising one or more strains of living probiotic bacteria. Any living probiotic bacteria suitable for human administration may be used. For example, probiotic bacterial strains may be selected from the genus Lactobacillus or Bifidobacterium. In one embodiment, the living bacterial strains that may be used include, but are not limited to B. animalis, B. adolescentis, B. bifidum, B. breve, B. lactis, B. longum, B. infantis. L. acidophilus, L. brevis, L. bulgaricus, L. casei, L. crispatus, L. fermentum, L. helveticus, L. lactis, L. gasseri, L. paracasei, L. plantarum, L. reuteri, L. rhamnosus, L. salivarius, S. thermophilus, E. faecium. In another embodiment, the probiotic formulation may include a combination of such living bacterial strains. In one embodiment, the probiotic formulation may be a blend of living bacterial strains of B. bifidum, B. breve, B. lactis, B. longum, B. infantis, L. acidophilus, L. brevis, L. bulgaricus, L. casei, L. gasseri, L. paracasei, L. plantarum, L. reuteri, L. rhamnosus, L. salivarius, and known as the Doctor's Biome Colon Health™ (DBCH). Each living bacterial strain present in the liquid juice medium is present in substantially the same percentage.

In one embodiment, the probiotic formulation of the method may be in the form of a juice beverage suitable for oral administration. The probiotic formulation may be a dietary supplement. The beverage may comprise living bacterial strains dispersed in a liquid medium. The beverage may be in the form of a sterilized liquid fruit and vegetable juice medium. The sterilized liquid fruit and vegetable juice medium may include, but is not limited to, mint juice, cucumber juice, apple juice, lettuce juice, kale juice, celery juice, lemon juice, and mixtures thereof. The sterilized liquid fruit and vegetable juice medium may be organic and free of chemicals, pesticides, and/or additives. The probiotic formulation of the invention may include one or more prebiotics. Examples of prebiotics used in the prebiotic formulation include but is not limited to inulin, maltodextrin, starch, and/or mixtures thereof. In some embodiments, the probiotic formulation of the method may comprise no-added sugar in the range of 5%-15%. For example, the probiotic formulation of the method may comprise no-added sugar concentration of 5%, 6%, 7%, 8%, 9%, 10%, 11%, 12%, 13%, 14%, or 15%.

In one embodiment, the probiotic formulation of the method may be formulated to provide between 1 billion and 200 billion colony forming units in a serving. The probiotic formulation may comprise between 1 billion and 200 billion colony forming units of pre-hydrated, actively living bacterial strains in the sterilized liquid fruit and vegetable juice medium. In one embodiment, there may be between 1 billion and 200 billion colony forming units of pre-hydrated, actively living bacterial strains in a 2-ounce bottle of the sterilized liquid fruit and vegetable juice medium.

The probiotic formulation of the method comprises bioactive compounds released by the living bacterial strains. In addition to competitive exclusion of C. difficile or C. perfringens, the bioactive compounds may reduce the pH of the user's gut to an acidic range unfavorable for spore germination and/or growth of C. difficile or C. perfringens, thereby preventing and/or ameliorating Clostridium difficile infection (CDI) or C. perfringens infection.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

As will be recognized by those of ordinary skill in the pertinent art based on the teachings herein, numerous changes and modifications may be made to the above-described and other embodiments of the invention without departing from its scope. Accordingly, this detailed description of preferred embodiments is to be taken in an illustrative as opposed to a limiting sense.

The following are examples exhibiting inhibitory effect of the probiotic formulation of the invention.

### Example 1

A test was performed to determine the fate of C. difficile in the presence of the probiotic formulation disclosed in the present invention. 2-ounce bottles of the Colon Health Probiotic Juice (probiotic formulation) of the present invention were provided to Eurofins Microbiology Laboratories. Upon receipt, the samples were stored at ambient temperature.

### Materials and Methods:

### 1. Preparation of Stock Solution:

C. difficile strain (ATCC 9689) was used in this study by isolating a colony of C. difficile and subculturing to Trypticase-Peptone-Glucose-Yeast-Extract (TPYGE) broth. The culture was incubated anaerobically at 37° C./99° F. for 18 to 20 hours. Following incubation, the culture was stored in an anaerobic chamber overnight at 4° C./40° F. The inoculum was then removed from the refrigerator and allowed to reach ambient temperature before use.

### 2. Preparation of Test Sample and Control Sample:

2-ounce bottles of the probiotic formulation (DBCH) of the present invention were then composited and homogenized into one sample, centrifuged at 4500 RPM for 15 minutes. This sample was called “test sample.” Sterile 0.1% peptone water was used as the “control sample.”

### 3. Evaluation of Inoculated Samples and Test Control Samples:

The stock inoculum of C. difficile was vortexed for 5 seconds before 1.0 ml of the stock inoculum was transferred to a 9 ml tube containing test sample or control sample. Each tube was serially diluted with either the test or control samples. The dilutions were then plated onto TPGYE Agar. The plates were incubated anaerobically for 72 hours at 37° C./99° F. Colonies were then counted based on characteristic colony morphology typical to C. difficile on TPGYE agar.

### Results:

The pH value of the pre-centrifuge the probiotic formulation sample was 3.24. Total counts recovered on TPGYE from serially diluted test samples and control samples are reported in Table 1. The population levels of C. difficile recovered in the test sample and control sample were <10 CFU/ml and 1.8×105 CFU/ml, respectively.

TABLE 1
Total counts recovered on TPGYE from serially diluted
test samples and control samples. Total counts are
reported as CFU/ml.

| | Total Counts | Total Counts |
|---|---|---|
| | Recovered | Recovered |
| | from Serially | from Serially |
| | Diluted Test | Diluted Con- |
| | Samples | trol Samples |
| | (Colon Health | (Peptone |
| Dilution | Probiotic Juice) | Water) |
| 1:10 | 0 | TNTC1 |
| 1:100 | 0 | TNTC1 |
| 1:1,000 | 0 | 178 |
| 1:10,000 | 0 | 18 |
| 1:10,000 | 0 | — |
| 1:100,000 | 0 | — |

¹Too numerous to count.

### Conclusion:

The results of the study indicated that C. difficile was not recovered from the samples containing the probiotic formulation (pH=3.24) at any of the dilutions tested.

### Example 2

### 1. Culture Propagation and Strain Verification:

A sterile inoculating loop of C. difficile (ATCC 9689) was streaked onto a Reinforced Clostridial agar slant, broth tubes, and two Sheep Blood agar plates. The Reinforced Clostridial agar slant, broth tubes and one Sheep Blood agar plates were incubated at 36-38° C. for 48±2 hours under anaerobic conditions. The other Sheep Blood agar plate was incubated for 36-38° C. for 48±2 hours under aerobic conditions, which served as a contamination check. After incubation, the agar plates and the broth tubes were checked for colony growth and turbidity. The Sheep Blood agar plated was biochemically identified using the VITEK ANC card to verify purity.

### 2. Preparation of Inoculum:

A well isolated colony from the Sheep Blood agar late was used inoculate a Reinforced Clostridial broth. The inoculated Reinforced Clostridial broth was then incubated anaerobically at 36-38° C. for 18 to 20 hours. Following incubation, the culture was stored overnight at 3±1° C.

### 3. Preparation of Sample:

Six, individually packaged, intact 2-ounce of DBCH glasses provided to Certified Labs were combined into a sterile 500 mL glass bottle under a laminar air flow hood. The bottle was then swirled gently and stored at 20-25° C. until it was used for testing. At that time, the composited product was homogenized. 10 mL was then pipetted into a test tube for a before and after comparison with the centrifuged product. Following centrifugation at 4500 RPM for 15 minutes, the supernatant was filtered through a 0.22 μm filter to ensure that the supernatant was free of cells. 10 mL portion of the supernatant was pipetted into a test tube and used as a test product sample. 9 mL diluent tubes were used for its ten-fold serial dilution. Sterile 0.1% Peptone water was inoculated and used as control.

### 4. Test Procedure:

### Control Test Procedure:

The inoculum used for the sample test procedure was vortexed for 5 seconds. 1 mL of inoculum was added to a 9 mL tube of 0.1% Peptone Buffer, and vortexed for 5 seconds, which is the 10⁻¹ dilution tube. 1 mL from the 10⁻¹ dilution tube was transferred in duplicate into sterile Petri Dishes. 1 mL was transferred from the same pipet into a second 9 mL tube containing 0.1% Peptone Buffer for the 10⁻² dilution tube, and then vortexed for 5 seconds. 1 mL is then transferred from the 10⁻² in duplicate into sterile Petri Dishes. 1 mL was transferred into a third 9 mL containing 0.1% Peptone Buffer for the 10⁻³ dilution tube and vortexed for 5 seconds. Such serial dilution was repeated until a 10⁻⁸ dilution was reached. Approximately 20 mL of Reinforced Clostridial agar (at 44-46° C.) was poured into the Petri Dishes and gently swirled to disperse the inoculum throughout the agar. Once the agar solidified, 15 mL of Reinforced Clostridial agar was overlaid onto each plate. The agar plates were then incubated anaerobically in an upright position at 35-37° C. for 18-24 hours.

### Sample Test Procedure:

The refrigerated anaerobic chamber containing inoculum was brought to ambient temperature prior to inoculation. The inoculum was vortexed for 5 seconds to homogenize. 1 mL of the inoculum was transferred to a 9 mL tube of supernatant test sample and vortexed for 5 seconds, which served as 10⁻¹ dilution tube. 1 mL from the 10⁻¹ dilution tube was transferred in duplicate into sterile Petri Dishes. With the same pipet, 1 mL was transferred into a second 9 mL tube containing the supernatant test sample for the 10⁻² dilution tube and vortexed for 5 seconds. 1 mL from the 10⁻² dilution tube was transferred in duplicate into sterile Petri Dishes. With the same pipet, 1 mL will be transferred into a third 9 mL tube containing the supernatant test sample for the 10⁻³ dilution tube and vortexed for 5 seconds. Serial dilution was repeated until a 10-8 dilution plate was reached. Approximately 20 mL of Reinforced Clostridial agar (at 44-46° C.) was poured into the Petri Dishes and gently swirled to disperse the inoculum throughout the agar. Once the agar solidified, 15 mL of Reinforced Clostridial agar was overlaid onto each plate. The agar plates were then incubated anaerobically in an upright position at 35-37° C. for 18-24 hours.

### Results:

Following incubation, photographs of the serially diluted agar plates were taken. The control plates visually showed presence of C. difficile colony forming units. The plates which exhibited less than 300 colonies were enumerated and representative colonies for Clostridium difficile were biochemically identified via VITEK ANC. following incubation, photographs were taken of the serially diluted agar plates of the sample to visually show absence of Clostridium difficile colony forming units due to the inhibitory effect of the DBCH bioactive compounds

Colony counts are tabulated below:

| | Control | | Sample | |
|---|---|---|---|---|
| Dilution | Rep 1 | Rep 2 | Rep 1 | Rep 2 |
| 10^1 | TNTC | TNTC | 0 | 0 |
| 10^2 | TNTC | TNTC | 0 | 0 |
| 10^3 | TNTC | TNTC | 0 | 0 |
| 10^-4 | TNTC | TNTC | 0 | 0 |
| 10^-5 | TNTC | TNTC | 0 | 0 |
| 10^-6 | 62 | 40 | 0 | 0 |
| 10^-7 | 3 | 1 | 0 | 0 |
| 10^-8 | 0 | 0 | 0 | 0 |

*TNTC: Too numerous to count.

### Example 3

Growth inhibition of C. perfringens is examined in the presence of the probiotic medical food (identified as MF-MS for testing) of the invention. For preparation of stock solution, two epsilon-toxin producing strains of C. perfringens from American Type Culture Collection: ATCC 3626 (Type B) and ATCC 3631 (Type D) are selected.

For preparation of test samples and control samples, individual 2-ounce bottles of MF-MS are composited and homogenized to one sample, and then centrifuged at 4500 RPM for 15 minutes to prepare the “test sample.” PYGS broth for C. perfringens is used as the “control sample”. The “test sample” is aliquoted into two portions and each portion is bulk-inoculated with one of the inoculums (either C. perfringens ATCC 3626 or ATCC 3631) at a target level of ~4 log CFU/ml. Immediately following inoculation, the samples are mixed to ensure even distribution of the inoculum.

PYGS broth is inoculated with C. perfringens ATCC 3626 and ATCC 3631 separately, as the “control samples.” The inoculated test samples and control samples are incubated anaerobically at 30° C./86° F. for up to 5 days. At each sampling time, the inoculated test samples and control samples are removed from the incubator and 3×1 ml of sample is removed from the inoculated bulk sample, serially diluted with 0.1% peptone water, and enumerated for the target organisms. The appropriate dilutions are then plated onto Trypticase-Peptone-Glucose-Yeast Extract (TPGYE) agar. The plates are incubated anaerobically for 72 hours at 30° C./86° F. Colonies are counted based on characteristic colony morphology typical to C. perfringens on TPGYE agar. All microbiological data is reported as CFU/ml. The limit of detection via plating method is 10 CFU/ml.

### Results

While on Day 0, the ATCC 3626 and ATCC 3631 population in the test sample are almost equal to that in the control sample, their growth is inhibited in the test sample. In contrast, ATCC 3626 and ATCC 3631 in the control sample showed a typical growth curve for microorganisms.

1. A probiotic formulation comprising:
a liquid medium comprising a plurality of living probiotic bacterial strains of the genus Bifidobacterium and genus Lactobacillus in a sterilized liquid fruit and vegetable juice medium, wherein the plurality of living probiotic bacterial strains of the genus Bifidobacterium comprise at least one species selected from among B. bifidum, B. breve, B. lactis, B. longum, B. infantis and wherein the plurality of living probiotic bacterial strains of the genus Lactobacillus comprise at least one species selected from among L. acidophilus, L. brevis, L. bulgaricus, L. casei, L. gasseri, L. paracasei, L. plantarum, L. reuteri, L. rhamnosus, L. salivarius;
wherein each living bacterial strain is present in the liquid medium in substantially a same percentage; and
wherein the probiotic formulation has a sugar content within a concentration range of about 5% to about 15%.

2. The probiotic formulation according to claim 1, wherein the plurality of living probiotic bacterial strains of the genus Bifidobacterium comprise the species B. bifidum, B. breve, B. lactis, B. longum, B. infantis and the living probiotic bacterial strains of the genus Lactobacillus comprise the species L. acidophilus, L. brevis, L. bulgaricus, L. casei, L. gasseri, L. paracasei, L. plantarum, L. reuteri, L. rhamnosus, L. salivarius.

3. The probiotic formulation according to claim 1, wherein the sterilized liquid fruit and vegetable juice medium includes at least one of mint juice, cucumber juice, apple juice, lettuce juice, kale juice, celery juice, lemon juice, and mixtures thereof, wherein all the juices are organic without pesticides, chemicals or additives.

4. The probiotic formulation according to claim 1, wherein the probiotic formulation further comprises a prebiotic.

5. The probiotic formulation according to claim 1, wherein the formulation is a drinkable beverage with a desirable shelf-life while also maintaining viability of the bacterial strains without the need of refrigeration or cooling of the beverage.

6. The probiotic formulation according to claim 1, wherein the formulation comprises from 1 to about 200 billion Colony Forming Unites (CFUs) of pre-hydrated, actively living probiotics in a 2 fluid ounce bottle of a juice beverage at the time of manufacture.

7. The probiotic formulation according to claim 1, wherein the formulation inhibits the growth of undesirable bacteria and pathogens in a user's gut and helps ameliorate Clostridium difficile infection (CDI) and/or multiple sclerosis (MS).

8. The probiotic formulation according to claim 1, wherein the selected plurality of living probiotic bacterial strains release bioactive compounds into the selected sterilized liquid fruit and vegetable juice medium and is configured to inhibit growth of C. difficile and/or C. perfringens.

9. The probiotic formulation according to claim 8, wherein the effect of bioactive compounds being released reduces the pH of the user's gut to an acidic range unfavorable for spore germination and/or growth of C. difficile and/or C. perfringens.

10. The probiotic formulation according to claim 1, wherein the formulation is a dietary supplement or a medical food.

11. A method of improving gut health of an individual with C. difficile, the method comprising:
providing to an individual with C. difficile a probiotic formulation to reduce or inhibit growth of C. difficile in the individual, thereby improving gut health of the individual, wherein the probiotic formulation comprises:
a liquid medium comprising a plurality of living probiotic bacterial strains of the genus Bifidobacterium and genus Lactobacillus in a sterilized liquid fruit and vegetable juice medium, wherein the plurality of living probiotic bacterial strains of the genus Bifidobacterium comprise at least one species selected from among B. bifidum, B. breve, B. lactis, B. longum, B. infantis and wherein the plurality of living probiotic bacterial strains of the genus Lactobacillus comprise at least one species selected from among L. acidophilus, L. brevis, L. bulgaricus, L. casei, L. gasseri, L. paracasei, L. plantarum, L. reuteri, L. rhamnosus, L. salivarius;
wherein each living bacterial strain is present in the liquid medium in substantially a same percentage; and
wherein the probiotic formulation has a sugar content within a concentration range of about 5% to about 15%.

12. The method according to claim 11, wherein the plurality of living probiotic bacterial strains of the genus Bifidobacterium comprise the species B. bifidum, B. breve, B. lactis, B. longum, B. infantis and the living probiotic bacterial strains of the genus Lactobacillus comprise the species L. acidophilus, L. brevis, L. bulgaricus, L. casei, L. gasseri, L. paracasei, L. plantarum, L. reuteri, L. rhamnosus, L. salivarius.

13. The method according to claim 11, wherein the sterilized liquid fruit and vegetable juice medium includes at least one of mint juice, cucumber juice, apple juice, lettuce juice, kale juice, celery juice, lemon juice, and mixtures thereof, wherein all the juices are organic without pesticides, chemicals or additives.

14. The method according to claim 11, wherein the probiotic formulation comprises from 1 to about 200 billion Colony Forming Units (CFUs) of pre-hydrated actively living probiotics in a 2-ounce bottle of a juice beverage at the time of manufacture.

15. The method according to claim 11, wherein the selected plurality of living probiotic bacterial strains release bioactive compounds into the selected sterilized liquid fruit and vegetable juice medium and is configured to inhibit growth of C. difficile.

16. The method according to claim 15, wherein the effect of bioactive compounds being released reduces the pH of the user's gut to an acidic range unfavorable for spore germination and/or growth of C. difficile, thereby preventing and/or ameliorating Clostridium difficile infection (CDI).

17. The method according to claim 11, wherein in the step of providing to an individual with C. difficile a probiotic formulation, the probiotic formulation is a dietary supplement or a medical food.

18. A method of dietary management, inhibition or amelioration of multiple sclerosis (MS) in an individual, the method comprising:
providing to an individual a probiotic formulation to reduce or inhibit growth of C. perfringens in the individual, wherein the probiotic formulation comprises:
a liquid medium comprising a plurality of living probiotic bacterial strains of the genus Bifidobacterium and genus Lactobacillus in a sterilized liquid fruit and vegetable juice medium, wherein the plurality of living probiotic bacterial strains of the genus Bifidobacterium comprise at least one species selected from among B. bifidum, B. breve, B. lactis, B. longum, B. infantis and wherein the plurality of living probiotic bacterial strains of the genus Lactobacillus comprise at least one species selected from among L. acidophilus, L. brevis, L. bulgaricus, L. casei, L. gasseri, L. paracasei, L. plantarum, L. reuteri, L. rhamnosus, L. salivarius;
wherein each living bacterial strain is present in the liquid medium in substantially a same percentage; and
wherein the probiotic formulation has a sugar content within a concentration range of about 5% to about 15%.

19. The method according to claim 18, wherein the plurality of living probiotic bacterial strains of the genus Bifidobacterium comprise the species B. bifidum, B. breve, B. lactis, B. longum, B. infantis and the living probiotic bacterial strains of the genus Lactobacillus comprise the species L. acidophilus, L. brevis, L. bulgaricus, L. casei, L. gasseri, L. paracasei, L. plantarum, L. reuteri, L. rhamnosus, L. salivarius.

20. The method according to claim 18, wherein the sterilized liquid fruit and vegetable juice medium includes at least one of mint juice, cucumber juice, apple juice, lettuce juice, kale juice, celery juice, lemon juice, and mixtures thereof, wherein all the juices are organic without pesticides, chemicals or additives.

21. The method according to claim 18, wherein the probiotic formulation comprises from 1 to about 200 billion Colony Forming Units (CFUs) of pre-hydrated actively living probiotics in a 2-ounce bottle of a juice beverage at the time of manufacture.

22. The method according to claim 18, wherein the selected plurality of living probiotic bacterial strains release bioactive compounds into the selected sterilized liquid fruit and vegetable juice medium and is configured to inhibit growth of epsilon toxin producing C. perfringens.

23. The method according to claim 22, wherein the effect of bioactive compounds being released reduces the pH of the user's gut to an acidic range unfavorable for spore germination and/or growth of C. perfringens, thereby inhibiting and/or ameliorating MS.

24. The method according to claim 18, wherein in the step of providing to an individual a probiotic formulation, the probiotic formulation is a dietary supplement or a medical food.
